(19) United States
(12) Patent Application Publication
(10) Pub. No.: US 2003/0158536 A1
(43) Pub. Date: Aug. 21, 2003

(54) SYRINGE INTERFACING SHEATH AND FILLING AID

(76) Inventor: VAUGHN R. JENSEN, GENOLA, UT (US)

Correspondence Address: ANGUS FOXX III, 4093 N. IMPERIAL WAY, PROVO, UT, 84604-5386, US

(21) Appl. No.: 09/460,209

(22) Filed: Dec. 13, 1999

(51) Int. Cl. A61B 19/00

(52) U.S. Cl. 604/403

(57) ABSTRACT

A combined interfacing sheath to protect syringe needles, and an aid to filling syringes by connecting the syringe to the bottle. A plastic sheath fits snugly over the syringe needle hub, thus protecting the needle during shipping and handling. The opposite end of the sheath includes a sleeve that extends beyond the syringe needle. This provides additional protection for the needle and a self aligning guide for inserting the syringe needle into the bottle stopper.

### CROSS-REFERENCE TO RELATED APPLICATIONS

Not Applicable

### REFERENCE TO MICROFICHE APPENDIX

Not Applicable

### BACKGROUND OF THE INVENTION

Syringe needles are becoming smaller in diameter and sharper to the touch. This increases the difficulty of filling a syringe as well as the possibility of personal injury or damage to the needle during the filling process. For many who require self injection (such as insulin dependent diabetics), the difficulty is magnified.

Although there are numerous devices recommended and employed to aid in the problem of filling syringes, all require full implementation in the field. This invention is a dual-function device. First, it is a sheath that protects the needle during shipping and handling. Secondly, this device serves as an aid for filling the syringe during subsequent injections.

### BRIEF SUMMARY OF THE INVENTION

This invention is an interfacing sheath to protect syringe needles, and an aid to filling syringes by connecting the syringe to the bottle. A plastic sheath fits snugly over the syringe needle hub, thus protecting the needle during shipping and handling. The opposite end of the sheath includes a sleeve that extends beyond the syringe needle. This provides additional protection for the needle and a self aligning guide for inserting the syringe needle into the bottle stopper.

This interfacing sheath is made of plastic strong enough to anchor the two components. It serves as a guide, making it easier to insert the syringe into the bottle stopper, limiting the margin of error and potential damage to the needle. The sheath also secures the syringe to the bottle.

The length of the interface is such that it barely admits the needle into the bottle. This feature enables the user to recover almost all of the liquid in the bottle with minimal effort-thus avoiding waste.

The interfacing sheath is intended to be attached and shipped with the syringe and remain in place through the filling process. This helps protect the needle from damage and the user from possible injury from the sharp point of the syringe needle.

### BRIEF DESCRIPTION OF THE DRAWINGS

FIG. 1, 2 and 3, shows views of the syringe interfacing sheath and filling aid in relation with syringes and serum bottles. FIG. 1 and 2 show exploded views, FIG. 1 includes numbers for annotation of the invention.

### DETAILED DESCRIPTION OF THE INVENTION

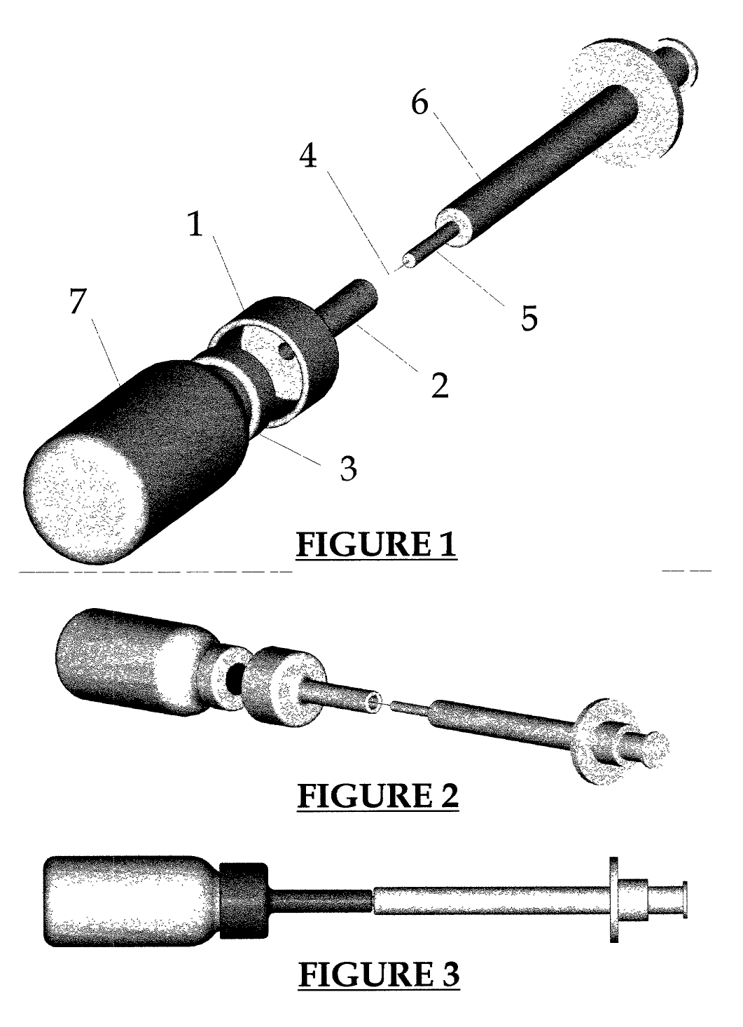

Referring to FIG. 1, this cylindrical interfacing sheath has two ends: the bottle end (1), a female fitting sized to fit standard serum bottles (7); and the syringe end (2), sized to the needle hub (5) of a syringe.

The syringe end (2) an interfacing sheath is adapted to the size of the needle hub (5). Where the needle sheath contacts the needle hub, the inside diameter is sized to match the outside diameter of the hub. This creates resistance sufficient to keep the interfacing sheath securely fastened to the syringe needle hub. The length of the needle end (2) is such that the syringe needle (4) extends into the bottle end (1) sufficient to allow correct needle penetration into the bottle stopper.

The bottle end of the sheath (1) has a cylindrical sleeve adapted to slide over the serum bottle cap (3). Where it contacts the bottle cap it is sized on the inside diameter the same as the outside diameter of the bottle cap, creating sufficient resistance to keep the bottle secure. The sleeve extends beyond the bottle cap (3) to the point where et nearly contacts the serum bottle (7). This provides protection for the needle (4), and also acts as a self aligning guide while inserting the needle into the bottle stopper. When the needle is fully inserted into the bottle stopper a physical connection is made between the syringe and the serum bottle thus completing the interfacing process. With the interfacing complete the filling of the syringe can be accomplished with minimal effort.

This invention is intended to be packaged with a tab over the bottle end, a cellophane wrapper or both. This provides a sanitary covering to prevent contamination until the syringe is used.

1. What I claim as my invention is a combined cylindrical interfacing sheath that protects syringe needles and provides an aid for filling syringes.

2. I claim the needle end of claim 1, a cylindrical sheath that attaches to the needle hub of syringes.

3. I claim the bottle end of claim 1, a cylindrical sleeve that aids in aligning and securing syringes with serum bottles.

4. I claim the protective tab that attaches to the bottle end of claim 1.
